(19) United States
(12) Patent Application Publication
(10) Pub. No.: US 2016/0250140 A1
(43) Pub. Date: Sep. 1, 2016

(54) METHOD FOR STERILIZING PLANT SEED, PRESERVATION METHOD, COSMETIC SEED, AND COSMETIC SET COMPRISING COSMETIC SEED

(71) Applicant: TOKO CO., LTD., Tokyo (JP)

(72) Inventor: Meiko Oh, Tokyo (JP)

(21) Appl. No.: 15/032,597

(22) PCT Filed: Oct. 28, 2013
(86) PCT No.: PCT/JP2013/006358

§ 371 Date: Apr. 27, 2016

(51) Int. Cl. A61K 8/97; A61Q 19/00; B65B 55/18; A61L 2/20

(52) U.S. Cl. CPC A61K 8/97; A61L 2/20; A61L 2/206; A61Q 19/00; B65B 55/18; A61K 2800/524; A61K 2800/87

(57) ABSTRACT

The present invention allows a plant seed that contains at least galacturonic acid and rhamnose as constituents to be reduced in a general bacterial count to a cosmetically acceptable level by placing the plant seed under reduced pressure, subsequently exposing the plant seed in bacteriocidal gas atmosphere with higher pressure than atmospheric pressure, and then placing the plant seed under reduced pressure. The plant seed of the present invention can form a pectic coat in water upon soaking in the water. The bacteriocidal in the present invention can be selected from any combination of ethylene oxide, ethylene glycol, ethylene chlorohydrin, and inert gas.

### BACKGROUND

1. Technical Field

The present invention relates to a cosmetic product using a natural material, more particularly, a disinfection method, a preservation method of a cosmetic composition using a plant seed, a cosmetic seed, and a cosmetic kit including the cosmetic seed thereof.

2. Background Art

Recently, development of chemical technologies has generated a variety of compounds with cosmetic effects and provided them in form of cosmetic compositions. As for the cosmetic effects, moisturizing, whitening, and other purposes and effects are known, and various novel compounds have been proposed and commercially available by means of chemical synthesis.

However, cosmetic composition rarely undergoes rigorous tests for a pharmacological action and a side effect as quasi drug, and thus is also known to have occurrence of various problems. Meanwhile, there is also a natural material known to have a cosmetic effect in literature. Notably, aqueous extraction from plant seeds of the family Acanthaceae is known to have a high moisturizing effect, and a kit using seeds of the genus Hygrophila, family Acanthaceae is known as Japanese Utility Model No. 3,176,415 (Patent document 1).

In use of natural materials such as plant seeds in the form of a cosmetic composition, the plant seeds are harvested from a plant. The seeds thus inevitably receive attachment of microbes thereon, and it is known that, when they are provided as-is for cosmetic use, some people may produce skin manifestations such as flare and itch due to the microbes attached onto the seeds. Therefore, sterilization must be required for utilizing plant seeds for cosmetic use.

Various methods are known for disinfection or sterilization of a plant seed, e.g., ozone gas treatment described in Japanese Patent Publication No. 2007-332069 (Patent document 1). In addition, gamma irradiation and the like are also known. These disinfection or sterilization methods are performed for the main purpose of improving long storage stability and seedling germination of seeds and the like. Therefore, they do not intend to disinfect or sterilize without change of any cosmetic property of a plant seed.

For the purpose of disinfection or sterilization of a plant seed, for example, the gamma ray irradiation or the ozone gas treatment is applied, the purpose of sterilization may be achieved. However, a method such as the gamma ray irradiation or the ozone irradiation may cause reduction in extractability of cosmetic constituents from the plant seed, thereby failing to achieve a cosmetic purpose such as moisturizing. Thus, even a sterilized or disinfected plant seed has often been unavailable for a cosmetic application as the predisinfection thereof and it has been difficult to sterilize or disinfect a natural material such as a plant seed without the reduction in any cosmetic property. Further, there has been needs of sterilization or disinfection methods that allows for preserving a sterile condition for a long period.

### CITATION LIST

Patent Literature 1: Japanese Utility Model No. 3,176,415

Patent Literature 2: Japanese Patent Publication No. 2007-332069

### SUMMARY OF THE INVENTION

### Technical Problem

Although various sterilization and disinfection of plant seeds have been examined so far, a method of sterilization or disinfection treatment of a plant seed up to the level that allows for the use of the plant seed as a cosmetic composition with a cosmetic property thereof has not been known yet. Further, a method of sterilization or disinfection that enables to preserve a sterile condition over a period required for distributing a product also has not been known yet.

The present invention has been made by contemplating the above-mentioned problems of the conventional arts, and an object of the present invention is to provide a sterilization method, preservation method and a cosmetic seed that allow for providing a cosmetic kit using a natural constituent from a plant seed without loss of any cosmetic property of the plant seed so as to provide the plant seed as a cosmetic composition.

Further, the present invention has an object to provide a cosmetic kit including the cosmetic seed.

### Solution to Problem

According to the present invention, there is provided a sterilization method of a plant seed that contains at least galacturonic acid and rhamnose as constituents, comprising placing the plant seed under reduced pressure, subsequently exposing the plant seed in bacteriocidal gas atmosphere with higher pressure than atmospheric pressure, and then placing the plant seed under reduced pressure so as to allow the plant seed to be reduced in bacterial count to a cosmetically acceptable level.

The plant seed of the present invention can form a pectic coat in water upon soaking in the water. The bacteriocidal in the present invention may be selected from the group consisting of ethylene oxide, ethylene glycol, ethylene chlorohydrin, and inert gas and any combination thereof.

In addition, according to the present invention, it is possible to provide a cosmetic seed which is a plant seed that contains at least galacturonic acid and rhamnose as constituents and form a pectic coat in water in case of soaking in the water, and further has a general bacterial count of 100 or less per 1 g. The plant seed may be a seed of a plant belonging to the family Acanthaceae.

The present invention may also provide a preserving method comprising containing the seed described above in a sealed package, and preserving a bacterial count that is cosmetically acceptable for the cosmetic seed.

Furthermore, according to the present invention it is possible to provide a cosmetic kit containing in an openable sealed package the cosmetic seed. The kit may further contain a constituent selected from hyaluronic acid, collagen, clay mineral, palm oil, alcohol, crystalline cellulose, polyhydric alcohol, almond oil, perfume, and castor oil, or combination thereof.

### BRIEF DESCRIPTION OF THE DRAWINGS

FIG. 1 shows a plot of the bacterial count and the processing time according to the embodiment.

### EMBODIMENTS FOR PRACTICING INVENTION

The present invention will hereafter be described with embodiments, but not limited to the embodiments below. The cosmetic composition of the present invention contains at least a sterilized or disinfected plant seed. The plant seed that has a nature of releasing a pectic composition upon soaking in water is available for the present invention. The plants available for the present invention may include, particularly, for example, the plant seed of the tribe Justicieae or Ruellieae, subfamily Acanthoideae, family Acanthaceae.

Genus belonging the tribe Justicieae may include the genus Pachystachys, Dicliptera, Peristrophe, Hypoestes, Justicia, Codonacanthus, Fittonia, Jacobinia, and particularly, the genus Justicia. Additionally, genus belonging the tribe Ruellieae include Hygrophila, Strobilanthes, Ruellia, Eranthemum, Hemigraphis, and particularly, the genus Hygrophila. The above plants may include a plant seed capable of releasing a pectic composition that contains at least garactulonic acid and rhamnose upon soaking in water.

The pectic coat is predicted to have a structure containing homogalacturonan, and rhamnogalacturonan-I or rhamnogalacturonan-III as the major constituents thereof, but not limited to, and also differs from fucoidan containing fucose as the major constituent thereof in that the polysaccharide thereof contains galactose as the major constituent.

Furthermore, the plant seeds other than those mentioned above, such as Hygrophila, Justicia Americana, Ruellia brevifolia, and Sanchezia noblis, which are genetically related species of Justicieae and Rullieae of the family Acanthaceae, are preferably available, as long as it can form a pectic coat in water.

The pectic coat may contain polysaccharides such as fructose, glucose, and galactose. Regularly, a plant seed (including a wild ones) is obtained from a fruit which is formed by growing a plant by soil culture, hydroponic culture, or the like, and maturing the plant. The seed may show adherence or inclusion of several bacteria referred to as general bacteria, since it is often difficult to render an environment where the plant is grown germ-free in both soil culture and hydroponic culture.

The bacteria such as general bacteria may produce flare or itch on some user depending on their skin environment, and it is not preferable to provide the plant seed as-is for cosmetic use. Therefore, it may be required to sterilize or disinfect the plant seed to reduce the general bacteria to a cosmetically acceptable level for providing as a cosmetic seed. Incidentally, one that may provide a cosmetic effect in the embodiment is pectic coat, which may be spontaneously released when the plant seed soaks in water.

The inventor has found that disinfection and sterilization treatment may affect on the formation of the pectic coat to complete the present invention. Various methods are known as methods of disinfection and sterilization for the plant seeds, such as gamma irradiation, ozone gas treatment, disinfection with acidic water, or gas disinfection. The inventor has made earnest examinations, and thereby found as one of the disinfection or sterilization methods; this disinfection method may allow to kill only bacteria to a cosmetically acceptable level without any damage on the polysaccharide of the plant seed to arrive at the present invention.

Among the disinfection or sterilization methods described above, gamma ray irradiation, ozone gas treatment, and the like were found to result in no formation of a coat at a degree acceptable to closely contact with a user's skin. Although the reason for this has not been identified yet, and also not limited to a certain reason, the inventor has supposed that the gamma ray irradiation as well as the ozone gas treatment etc. may degrade and/or oxidize the polysaccharides of the plant seed to reduce the capacity for formation of a pectic coat.

In addition, sterilization with acidic water is also available, but cannot be practically used for the present invention because as soon as soaking plant seeds in water, the plant seed may start to form a pectic coat. The most preferable sterilization or disinfection method according to one embodiment in the present invention is gas treatment. In particular, gas sterilization using ethylene oxide, ethylene glycol, ethylene chlorohydrin, and inert gas such as carbon dioxide, nitrogen or argon, and the gas mixture thereof has been found to allow for reducing bacterial count to a cosmetically acceptable level without decrease in the pectic coat releasing of the plant seed.

The gas sterilization treatment applicable in the present invention is found preferably for example, usage of Capox 20 (20% of ethylene oxide plus 80% of carbon dioxide) of −60 kPa to 60 kPa or less as treatment pressure; for about 1.7 hours to 20 hours, more preferably 1.7 hours to 17 hours, most preferably 2 hours to 5 hours as treatment time and from room temperature to 50° C. for treatment temperature. The reason to subject the negative pressure for the treatment pressure is for filling intra-seed with the treatment gas both to increase treatment effectiveness and to improve long storage stability.

Less than 1.7 hours of the treatment time did not provide sufficient sterilization effectiveness, and also more than 5 hours of the treatment time did preserve a substantially cosmetically acceptable level of bacterial count and appear to tend to decrease an ability for formation of a pectic coat. The treatment temperature less than room temperature may not show preferable influences on sterilization and/or disinfection effectiveness, and may generate a problem in increasing device cost. The treatment temperature of 50° C. or more might also denature the plant seed and thus may not be preferable. The number of sterilization treatments is not particularly limited, but they can be repeated once to about ten times with consideration of treatment efficiency.

A cosmetic seed in the embodiment means a plant seed with general bacterial counts cosmetically acceptable. In the present invention, the cosmetic seed particularly refers to a plant seed to which the sterilization treatment described above is applied or which is obtained from a plant cultivated in a germ-free condition such as a plant factory. Cosmetically acceptable general bacterial count refers to the general bacterial count less than 100 cells/g or less. The general bacterial count can be defined, for example, as the bacterial count that is determined by culturing under 30-35° C. of culture temperature for 3-5 days using soybean-casein-digest agar medium (SCDLP agar medium) and counting with a colony counter.

The cosmetic seed described above can be maintained in a germ-free condition by sealing and preserving in a sealed package with filling of germ-free dry gas such as air, nitrogen gas, or argon gas. Further, a cosmetic kit of one of the embodiment can be a form in which the cosmetic seed is sealed in the sealed package described above. In using the cosmetic kit, a user may open a sealed package, soak the cosmetic seed contained therein in water to form a pectic coat. A preservation method of the present invention can maintain the sterilization level of the cosmetic seed to a cosmetically acceptable level over three to five years.

Subsequently, the formed pectic coat may be squeezed to eliminate water, put it as a cosmetic pack onto a part needed to be moisturized on face or body, and remove it after a predetermined time duration, and thereafter a desired part may be cared by a cosmetic treatment. Furthermore, in one of the embodiment, the cosmetic seed can be sealed suitably with an already-known additive such as hyaluronic acid, collagen, clay mineral, palm oil, alcohol, crystalline cellulose, polyhydric alcohol, almond oil, perfume, and castor oil, to be provided as a cosmetic kit.

Hereafter, the invention will be illustrated by reference to specific examples, but the invention is not limited to the embodiments described below.

### EXAMPLES

### Plant Seed

As for plant seeds, seeds of Hygrophila salicifolia, genus Hygrophila, family Acanthaceae were used. The content of the plant seeds used is listed in Table 1 below. As can be seen in Table 1 below, the plant seeds contains galacturonic acid and rhamnose, and may provide a pectic coat in water, Incidentally, the analysis for Table 1 below was performed in Japan Food Laboratories (52-1, Motoyoyogi-cho, Shibuya-ku, Tokyo) by request.

TABLE 1
Test Result(s)

| Test Item | Result | QL | N | M |
|---|---|---|---|---|
| Moisture | 7.8 g/100 g | — | | 1 |
| Protein | 20.7 g/100 g | — | 1 | 2 |
| Fat | 25.6 g/100 g | — | | 3 |
| Ash | 5.2 g/100 g | — | | 4 |
| Available carbohydrate | 4.8 g/100 g | — | 2 | |
| Dietary fiber | 35.9 g/100 g | — | | 5 |
| Glucuronic acid | Not detected | 0.2 g/100 g | 3 | 6 |
| Fructose | 1.2 g/100 g | — | 4 | 6 |
| Glucose | 8.6 g/100 g | — | 3 | 6 |
| Rhamnose | 0.9 g/100 g | — | 3 | 6 |
| Galactose | 3.0 g/100 g | — | 3 | 6 |
| Galacturonic acid | 1.3 g/100 g | — | 3 | 6 |

QL: Quantitation limit
N: Notes
M: Method
Notes
1: Nitrogen-to-protein conversion factor: 5.30.
2: Formula according to Notification No. 176 of 2003, issued by the Ministry of Health, Labour and Welfare: Available carbohydrate = 100 − (Moisture + Protein + Fat + Ash + Dietary fiber).
3: Acid hydrolysis was performed before measurement. Hydrolysis conditions: stirred for 1 hour at room temperature in 72% sulfuric acid and autoclaved (121° C.) for 1 hour in 4% sulfuric acid.
4: Acid hydrolysis was performed before measurement. Hydrolysis conditions: 2.3% hydrochloric acid at 65° C. for 15 minutes.
Method
1: Air oven method
2: Kjeldahl method
3: Soxhlet extraction method
4: Ashing method
5: Enzymatic-gravimetric method
6: HPLC

### Gas Sterilization

Gas sterilization was performed with GMS-0012-SW-AJ (12M³), an EOG sterilizer, using Capox 20 with a composition of 20% of ethylene oxide and 80% of carbon dioxide as the sterilization gas. Plant seeds with different sterilization level were obtained by setting prevacuum and postvacuum to −0.085 mPa; treatment pressure to 0.110 MPa; treatment temperature to 45° C.; treatment time to 1.5 hours, 2 hours, and 5 hours; and the numbers of the treatment to three times.

### Bacterial Counting

Bacterial count was determined by culturing under 30-35° C. of culture temperature for 3-5 days using soybean-casein-digest agar medium (SCDLP agar medium) and counting with a colony counter.

### Evaluation for Cosmetic Availability

The gas-sterilized cosmetic seeds had been sealed/preserved in a sterilized, sealed package for more than several weeks, and then the sealed package was opened to recover the sterilized plant seeds. The obtained plant seeds were soaked in tap water, resulting in successfully extracting a sufficient amount of pectic coats. The pectic coats was attached on the skin of the subject as a pack preparation, left for about 20-30 minutes, and then peeled off from the skin. There was neither flare, rash or the like on the post-peeling skin, nor itch in the subject.

The results described above have shown that the cosmetic seeds were maintained for a long period, suggesting that a cosmetic seed of the present invention does not lose its availability even through a sufficiently long channel of distribution.

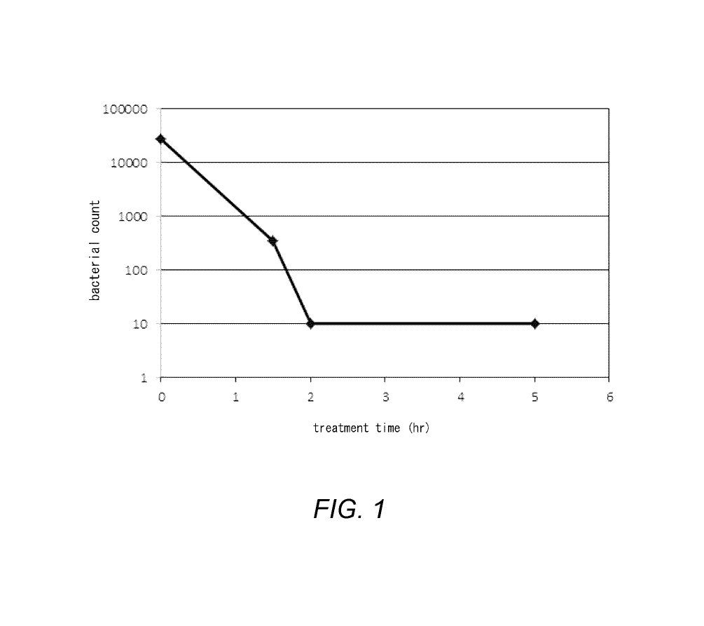

FIG. 1 shows the result of a plot of the bacterial count on the plant seed to treatment time. For comparison, the bacterial count of the hygrophila seeds without the gas treatment is jointly plotted.

As shown in FIG. 1, one of the gas sterilization treatments in the invention for at least 1.5 hours excutes sterilization but still do not achieve to 100 cells/g, which is a criteria for cosmetics and cosmetic use. In contrast, the gas sterilization treatment for 2.0 hours shows a remarkable reduction in the bacterial count to result in about 10 cells/g or less, which is sufficiently within the cosmetically acceptable range.

### Comparative Example

In Comparative example 1, the pre-sterilization general bacterial count of the hygrophila seeds used in Example was measured as the same condition as Example, resulting in 28000 cells/g, which was not suitable to employ as-is for cosmetic use.

In Comparative example 2, the hygrophila seeds similar to those used in Example was sterilized by gamma-ray irradiation and then subjected to general bacterial counting, showing that the general bacterial count reduced to the same level as that of Example, but that despite soaking in water, the sterilized seeds did not form a sufficient amount of pectic coats, and thus was unable to use as a cosmetic pack. The reason for this has not been cleared yet, but it can be supposed that the gamma-ray irradiation degraded polysaccharides such as galacturonic acid and rhamnose to lead to lack of the pectic coat formation.

Moreover, in Comparative example 3, the plant seeds similar to those used in Example was unsterilized, soaked as-is in tap water, and then applied to the subject as a pack in the similar manner as Example, resulting that the subject had an itch on the skin, and also was sometimes observed to exhibit skin inflammation due to bacteria, thereby failing to be applicable as-is for cosmetic use.

### INDUSTRIAL APPLICABILITY

As described above, according to the present inventions, constituents extracted from a natural plant seed can be employed for cosmetic use, and furthermore, sterilization method and preservation method of a cosmetic composition that is ecologically preferable, commercially distributable, and has less burden on a user; a cosmetic seed thereof; and a cosmetic kit including the cosmetic seed can be provided.

1. A sterilizing method of plant seeds that contains at least galacturonic acid and rhamnose as constituents, the method comprising:
placing the plant seeds under a reduced pressure;
subsequently exposing the plant seeds to a bacteriocidal gas atmosphere with a pressure higher than an atmospheric pressure; and
then placing the plant seed under the reduced pressure so as to allow the bacterial count of the plant seeds to reduce to a cosmetically acceptable level.

2. The sterilizing method according to claim 1, wherein the plant seeds forms a pectic coat in water upon soaking in the water.

3. The sterilizing method according to claim 1, wherein the bacteriocidal gas is selected from the group consisting of ethylene oxide, ethylene glycol, ethylene chlorohydrin, inert gas and any combination thereof.

4. A cosmetic seed comprising at least galacturonic acid and rhamnose as constituents and a pectic coat, wherein the cosmetic seed is a plant seed that has a general bacterial count of 100 or less per 1 g.

5. The cosmetic seed according to claim 4, wherein the plant seed is a seed of a plant belonging to the family Acanthaceae.

6. A preserving method comprising containing the cosmetic seed as set forth in claim 4 in a sealed package, and preserving a bacterial count that is cosmetically acceptable for the cosmetic seed.

7. A cosmetic kit comprising the cosmetic seed as set forth in claim 4 in an openable sealed package.

8. The cosmetic kit according to claim 7, further comprising a constituent selected from the group consisting of hyaluronic acid, collagen, clay mineral, palm oil, alcohol, crystalline cellulose, polyhydric alcohol, almond oil, perfume, castor oil, and a combination thereof.
